(19) United States
(12) Patent Application Publication
(10) Pub. No.: US 2009/0292208 A1
(43) Pub. Date: Nov. 26, 2009

(54) Automated detection of asymptomatic carotid stenosis

(76) Inventors: R. Brooke Jeffrey, JR., Los Altos Hills, CA (US); Butrus T. Khuri-Yakub, Palo Alto, CA (US); Sandy A. Napel, Menlo Park, CA (US); Omer Oralkan, Santa Clara, CA (US)

Correspondence Address: LUMEN PATENT FIRM, 350 Cambridge, Suite 100, PALO ALTO, CA, 94306, US

(21) Appl. No.: 12/380,889

(22) Filed: Mar. 3, 2009

Related U.S. Application Data
(60) Provisional application No. 61/068,004, filed on Mar. 3, 2008.

(51) Int. Cl. A61B 8/06

(52) U.S. Cl. 600/454

(57) ABSTRACT

Peak blood velocity measurement for automated stenosis detection is provided. Ultrasound measurements of the peak blood velocity are corrected by a calculation of the Doppler angle, which exists from misalignment of the ultrasound transducer axis and the true blood velocity. The direction of the blood velocity and the Doppler angle are found by imaging a set of planar cross-sections of a blood vessel, such as the carotid artery, to obtain velocity maps of the blood flowing in the blood vessel. Peak blood velocity can be correlated with an amount of stenosis therefore accurate peak blood velocity measurements are necessary for medical diagnosis. Automated stenosis detection allows for implementation in many medical settings. A capacitive micromachined ultrasound transducer array is also provided to measure the planar cross-sectional images.

### CROSS-REFERENCE TO RELATED APPLICATIONS

This application claims priority from U.S. Provisional Patent Application 61/068,004 filed Mar. 3, 2008, which is incorporated herein by reference.

### FIELD OF THE INVENTION

The invention relates generally to stenosis detection. More particularly, the present invention relates to medical ultrasound for automated detection of artery stenosis.

### BACKGROUND

Stroke is a major contributor to mortality and is the third leading cause of death in the United States each year. About 85% of strokes are ischemic, of which most are due to atherosclerosis (i.e. they are caused by blocked arteries). Early detection of asymptomatic stenosis of the internal carotid artery can play a significant role in the prevention of stroke. Stenosis detection in the carotid artery, or another blood vessel, requires imaging techniques, such as computer tomography, magnetic resonance, and ultrasound. Oftentimes, particularly for asymptomatic individuals, only ultrasound techniques are viable due to the lack of ionizing radiation and the low cost of ultrasound compared to other imaging techniques.

The dominant method for detection of asymptomatic stenosis relies on measurements of peak blood velocities (PBVs) in a blood vessel as the narrowing of the blood vessel changes the blood velocity and causes rapid flow regions or jets. The peak blood velocities can be correlated with a percentage of stenosis, for example a PBV greater than 125 cm/s in the internal carotid artery is generally associated with about 50% stenosis. Measuring the peak velocities can be difficult because the measured velocities are functions of an angle, referred to as the Doppler angle, between the flow direction and the transducer axis of the ultrasound used to make the measurement.

FIG. 1 shows an example of an existing ultrasound device 130 used to image the carotid artery 110 of subject 120. Existing ultrasound devices, such as ultrasound device 130, transmit and receive ultrasound beams 140 to image a segment of the artery. Since the blood is moving, the received beam will be Doppler-shifted from the transmitted beam. This shift is used to measure the PBV in the artery. However, it is important to note that the Doppler-shift is only measured for the component of the blood velocity that is along the same axis as the ultrasound beams 140. FIG. 2 shows a magnified view of the carotid artery 110 having plaque 210 that partially constricts a portion of the artery 110. The axis of the ultrasound beams 140 is shown and, in the configuration shown in FIG. 2, a velocity v_{D}, with magnitude (speed) |v_{D}| and direction along the ultrasound beams 140, is measured by the ultrasound device 130. Generally, the true blood velocity v is in a different direction than the measured velocity v_{D}. |v|, |v_{D}| and the so called Doppler angle, Θ_{D}, are related by the Doppler equation:

$$\vert v \vert = \frac{\vert {v}_{D} \vert}{cos Θ} \qquad ( 1 )$$

Thus, in order to determine the true blood velocity in the direction of the blood vessel, the angle between the ultrasound beams and the direction of blood flow, Θ_{D}, must be determined by the operator of the ultrasound device 130. Inaccurate estimates of Doppler angle Θ_{D} can lead to inaccurate readings of the PBV, and thus, limit efficient detection of those at risk for stroke.

Existing techniques to correct for the Doppler angle Θ_{D} challenge the skills of the operator. In most practices, the ultrasound device 130 is manually operated by the operator to find the location of the PBV and to determine the Doppler angle Θ_{D} so that it can be accounted for in Eq. 1. To do this, the operator of the ultrasound device 130 must move the device with translations 150 and/or rotations 160, 170, and, while holding the transducer steady in the final orientation, must manipulate the location and angles of at least two cursors on a display in order to determine the Doppler angle. However, this determination can be extremely difficult and requires a highly skilled operator, who may not be readily available.

Furthermore, some existing techniques rely on three-dimensional or four-dimensional (with a time-varying component) ultrasound imaging of the blood vessel. These complicated imaging techniques are costly to obtain, process, and analyze, and require complicated electronics and software.

The present invention addresses at least the difficult problems of finding peak blood velocities and advances the art with a novel ultrasound imaging method and technique.

### SUMMARY OF THE INVENTION

The present invention is directed to a method for determining peak velocity of fluid flowing in a blood vessel. The method includes measuring a set of spatially proximate ultrasound images along the blood vessel, wherein the set of ultrasound images are measured with an ultrasound transducer, wherein each of the ultrasound images is of a planar cross-section, oriented along the ultrasound beam, of the blood vessel, and wherein each of the ultrasound images provides a time-varying Doppler-generated velocity map of the component of the fluid flow in the direction of the ultrasound beam; determining an uncorrected peak velocity of the fluid, wherein the uncorrected peak velocity is located at or near one of the planar cross sections; identifying a high velocity center in each of a plurality of the ultrasound images; calculating a Doppler angle based on the high velocity centers of the ultrasound images; and correcting the peak velocity based on the calculated Doppler angle. In an embodiment, the corrected peak velocity can be correlated with an amount of stenosis in the blood vessel.

In a preferred embodiment, a processor or computer is provided for automated peak velocity determination. The processor automatically implements any number of the method steps described herein, including calculating the Doppler angle based on the high velocity centers of the ultrasound images.

In a preferred embodiment, the ultrasound transducer is a two-dimensional transducer array that is divided into a number of transducer sub-arrays, wherein each of the sub-arrays measures one of the ultrasound images of the set of ultrasound images. The ultrasound transducer can also include multiple acoustic lenses, wherein each of the acoustic lenses corresponds with one of the sub-arrays, and wherein each of the acoustic lenses allows the planar cross-section imaged by the corresponding sub-array to transect the blood vessel at a transect angle. Preferably, the transect angle is less than 45 degrees. In an embodiment, the ultrasound transducer includes an array of capacitive micromachined ultrasound transducers (CMUTs).

In an embodiment, the method also includes fitting a curve based on the identified high velocity centers of the ultrasound images, wherein the fitted curve represents the direction of the fluid flow in the blood vessel. The high velocity center of each of the ultrasound images can be identified based on a velocity-thresholded centroid, a velocity center of mass, or a location having the approximately highest velocity in the velocity map of the ultrasound image. In an embodiment, the imaged blood vessel bifurcates, such as in the carotid artery, and multiple high velocity centers are identified in each of at least one of the ultrasound images. One or more curves can be fit to represent the bifurcation. In an embodiment, the planar cross-sections of the set of ultrasound images are approximately parallel.

The present invention is also directed to a device for determining peak velocity of fluid flowing in a blood vessel. The device includes an array of ultrasound transducers, wherein the array is divided into multiple sub-arrays, wherein each of the sub-arrays produces a planar cross-sectional ultrasound image, oriented along the ultrasound beam, of the blood vessel, and wherein each of the ultrasound images provides a time-varying Doppler-generated velocity map of the component of the fluid flow in the direction of the ultrasound beam of the blood vessel, wherein an uncorrected peak velocity of the fluid is determined from the ultrasound images, wherein the uncorrected peak velocity is located at or near one of the planar cross-sections produced by the sub-arrays, wherein a high velocity center is identified in each of the ultrasound images, wherein a Doppler angle is calculated based on the high velocity centers of the ultrasound images, and wherein the peak velocity is corrected based on the calculated Doppler angle. In an embodiment, the device also includes multiple acoustic lenses, wherein each of the acoustic lenses corresponds with one of the sub-arrays, and wherein each of the acoustic lenses allows the planar cross-section imaged by the corresponding sub-array to transect the blood vessel at a transect angle. Preferably, the elements of the ultrasound transducer array are CMUTS.

In an embodiment, the ultrasound transducers have an operating frequency, wherein an acoustic wavelength in tissue corresponds with the operating frequency of the ultrasound transducers, and wherein the elements of said array of ultrasound transducers are spaced at approximately half of the acoustic wavelength. In an embodiment for carotid examination, the ultrasound transducers operate at a frequency range within approximately 5-15 MHz.

### BRIEF DESCRIPTION OF THE FIGURES

The present invention together with its objectives and advantages will be understood by reading the following description in conjunction with the drawings, in which:

FIG. 1 shows an example of a prior art technique of imaging the carotid artery.

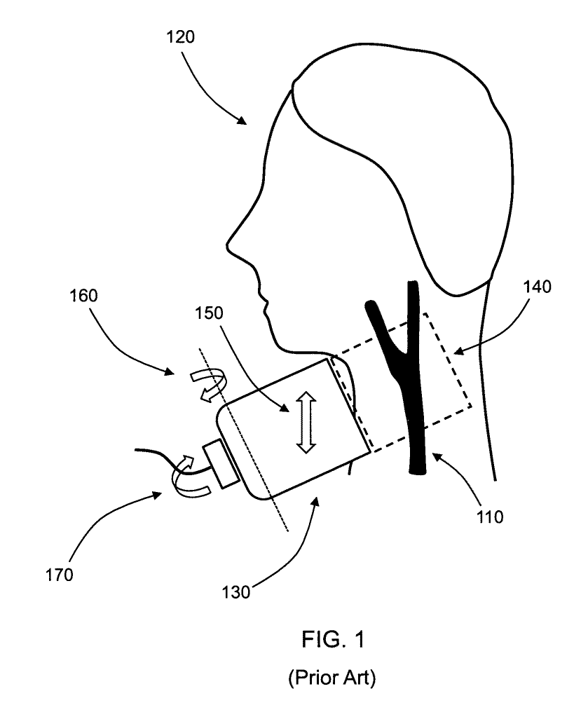

FIG. 2 shows the difference in the true blood velocity and the measured blood velocity due to the Doppler angle.

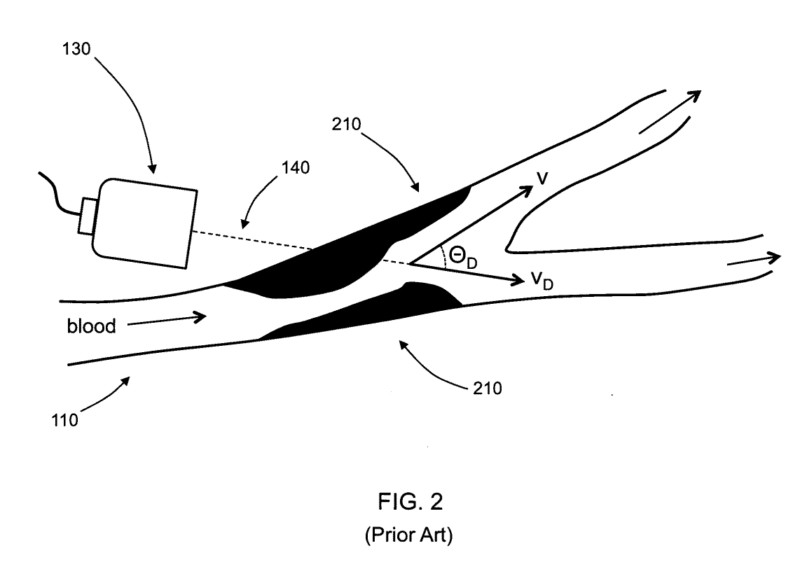

FIG. 3A-B shows an example of imaging the blood velocity at multiple cross-sections of a blood vessel according to the present invention.

FIG. 4 shows an example of calculating the Doppler angle based on the planar cross-sections imaged according to the present invention.

FIG. 5 shows a flow chart of an exemplary method of finding peak blood velocity according to the present invention.

FIG. 6A-B shows an example device having an array of ultrasound transducers for imaging the planar cross-sections according to the present invention.

### DETAILED DESCRIPTION OF THE INVENTION

The common occurrence of stroke, particularly due to asymptomatic carotid stenosis, indicates that detection of stenosis is an important and vital medical procedure. Unfortunately, accurate stenosis detection can be a daunting task. Though affordable ultrasound techniques have been developed, existing devices can only measure velocity longitudinal to the axis of the device, thereby causing ambiguous measurements of the peak blood velocity (PBV). Often, a skilled expert is required to accurately operate the device for true PBV measurement and determination of patients at risk for stroke.

The present invention is directed to the measurement of peak blood velocities in a blood vessel and using a calculation of the Doppler angle to correct the measured PBVs. Preferred embodiments of the present invention utilize planar cross-sectional images of the blood vessel to determine the Doppler angle and PBVs. The present invention does not require three-dimensional or four-dimensional imaging techniques, which can be financially or computationally expensive, have great hardware demands, and require extensive processing and analysis. However, it is noted that the present invention can also be used in combination with three-dimensional or four-dimensional ultrasound techniques.

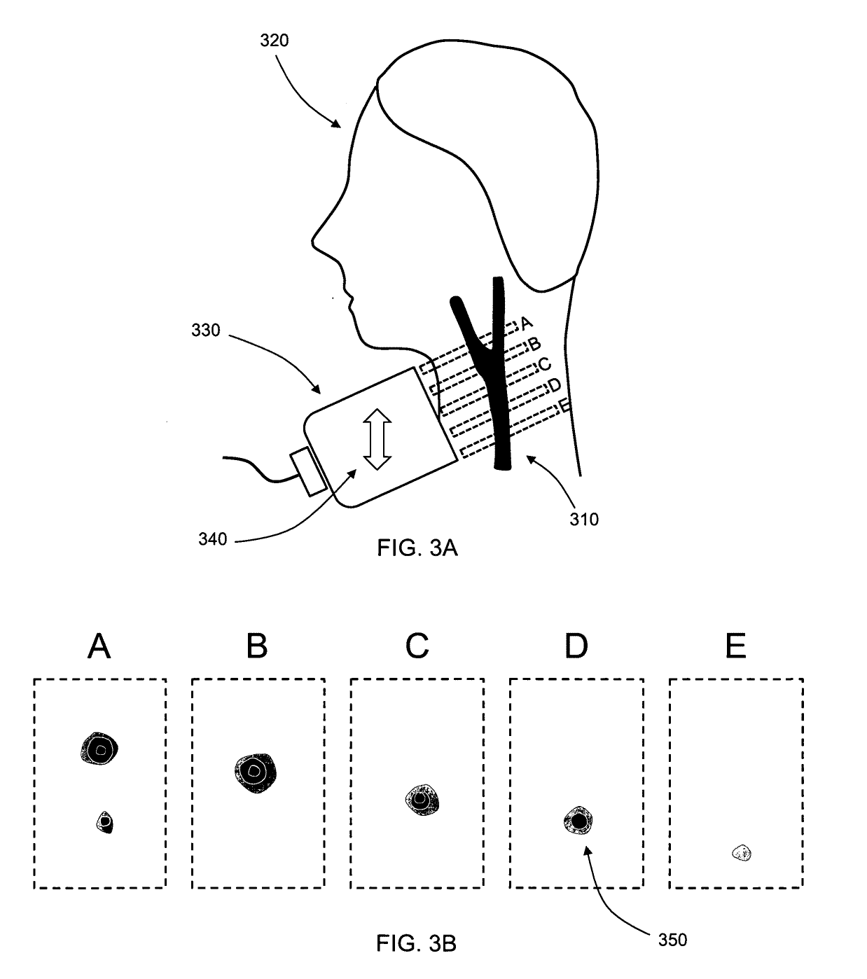

FIG. 3A shows an embodiment of the present invention for imaging the carotid artery 310 of a subject 320 with an ultrasound transducer 330. Though FIG. 3A and the examples herein describe imaging of the carotid artery 310 of a human subject 320, it is noted that the present invention can be applied to any blood vessel for any human or non-human subject. It is important to note that the ultrasound transducer 330 measures a set of spatially proximate ultrasound images A-E along the carotid artery 310. Each of the ultrasound images A-E is a planar cross-section of the carotid artery. Preferably, the ultrasound images A-E include at least all of the cross-sectional area of the blood vessel. Ultrasound imaging requires transmitting one or more ultrasound beams and receiving their reflections. The Doppler shifts of the transmitted and reflected beams are used to calculate a velocity map of the blood or other fluid in the image plane. In a preferred embodiment, the planar cross-sections transect the blood vessel with a transect angle less than approximately 45 degrees. Because Doppler shifts are accurately measured only when the blood flow has a significant component along the axis of the image planes, smaller transect angles are preferred.

FIG. 3B shows an example of the velocity maps imaged in FIG. 3A, where darker regions indicate greater velocities. By using velocity maps of the fluid imaged by the ultrasound transducer 330, the true PBV and Doppler angle Θ_{D} can be determined. High peak blood velocity can be correlated with stenosis (i.e., constriction of the blood vessel). An uncorrected PBV can be identified in the planar cross-section velocity maps A-E. The location of the uncorrected PBV is generally located at or near one of the planar cross-sections. In an embodiment, the operator of the ultrasound transducer 330 can move the transducer approximately along the blood vessel axis to find the location of the uncorrected PBV. This translational movement is indicated by the arrow 340. It is noted that the translational movement 340 is significantly simpler than the combination of translations and rotations 150-170 necessary to operate existing devices, where the operator attempts to determine the Doppler angle Θ_{D} simultaneously while finding the location of the PBV. Alternatively or in combination with translational movement 340 of the ultrasound transducer 330, the location of the uncorrected PBV can be found by extrapolation or interpolation of the set of velocity maps in the ultrasound images A-E.

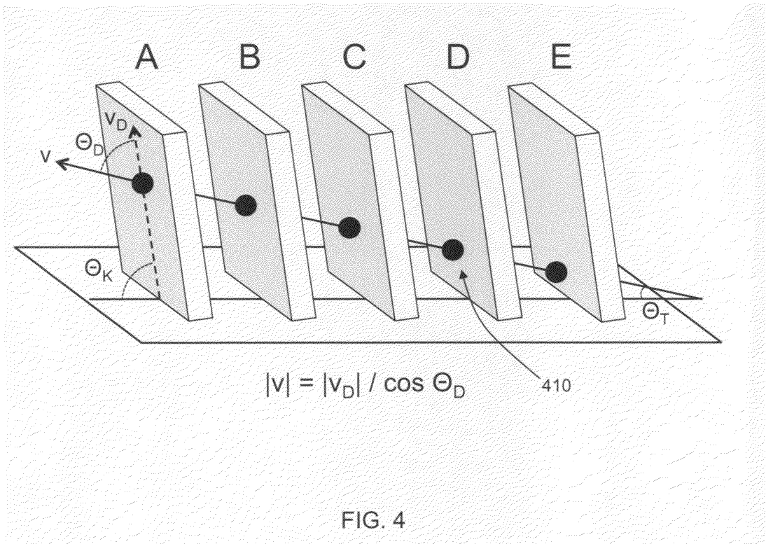

To determine the Doppler angle Θ_{D} for correcting the measured PBV, a high velocity center is identified in each of at least a subset of the ultrasound images A-E. The high velocity center can be identified by any technique, including based on a velocity-thresholded centroid, a velocity center of mass, or a location having the approximately highest velocity the velocity map. FIG. 4 shows the identified high velocity centers 410 for the ultrasound images A-E. The high velocity centers 410 are used to calculate the Doppler angle Θ_{D}. In a preferred embodiment, a curve is fitted for the high velocity centers, where the fitted curve represents the direction of blood flow in the vessel. The direction Θ_{T} of the peak blood velocity v is found in FIG. 4 by fitting a line to the high velocity centers 410. The Doppler angle Θ_{D} between v and v_{D} can then be calculated knowing the angle of construction of the ultrasound planes Θ_{K}, and Θ_{T}.

In an embodiment of the present invention, the fitted curve of the high velocity centers 410 is not a straight line. In certain embodiments, non-linear curves may more accurately represent the direction of blood flow, particularly when the imaged blood vessel has high curvature at the region of interest. When a non-linear curve is fit, the direction of the true PBV can be found based on a tangent of the curve at or near the location of uncorrected PBV.

It is noted that the present invention can also be applied to regions where the blood vessel bifurcates, such as in the carotid artery. FIGS. 3A-B show an example of imaging a bifurcating vessel. In an embodiment, bifurcations are accounted for by identifying multiple high velocity centers in each of at least one of the ultrasound images. For example, ultrasound image A of FIG. 3B shows that the blood vessel has likely bifurcated at a location between the planar cross-sections of image A and image B. When multiple high velocity centers are identified for each of at least one ultrasound image, one or more curves can be fitted to the high velocity centers of the set of ultrasound images. Different curves can be used to analyze the blood flow along the separate channels of the bifurcated vessel and can be used to calculate multiple Doppler angles.

In a preferred embodiment, the planar cross-sections of the set of ultrasound images are approximately parallel. Parallel cross-sections allow for simple calculation of the Doppler angle Θ_{D}. In alternative embodiments, the cross-sections imaged by the ultrasound transducer are not parallel. In these embodiments, the angles between the planar cross-sections are preferably known and can be accounted for in computing the vessel direction Θ_{T} required for calculating the Doppler angle Θ_{D}. It is noted that though the set of ultrasound images shown in the figures have five ultrasound images, any number of images (therefore, any number of planar cross-sections) can be used.

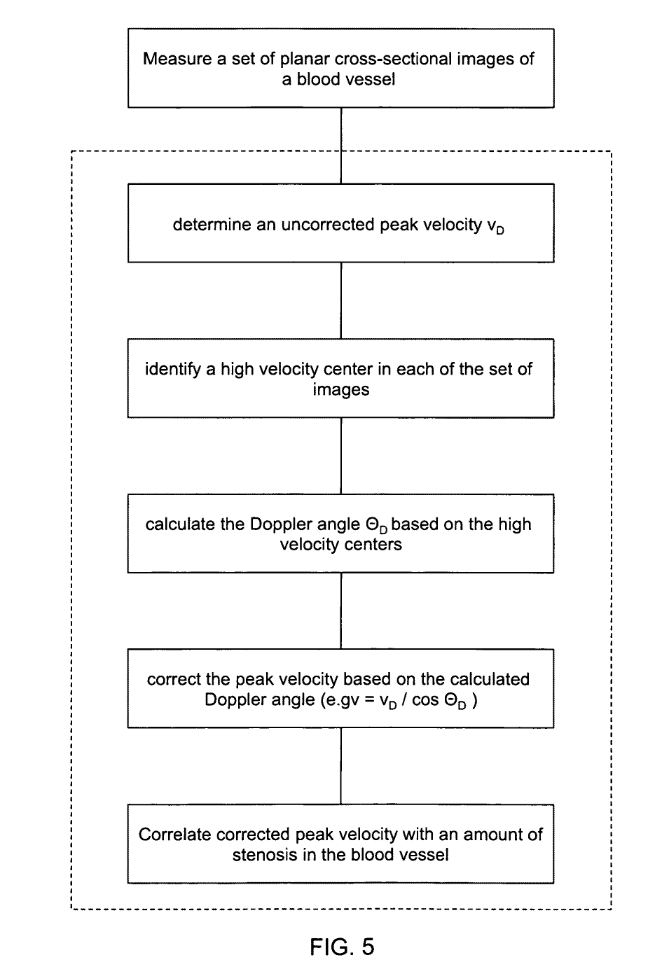

FIG. 5 shows a flow chart of an embodiment of the present invention. The dashed box encloses the steps that can be automated, i.e. without human intervention, though manual overrides are possible in certain embodiments. For example, a processor or computer is used to automatically implement one or more of the following steps: determine an uncorrected peak velocity, identify high velocity centers, calculate the Doppler angle Θ_{D}, correct the peak velocity based on the Doppler angle Θ_{D}, and correlate the corrected peak velocity with a degree of stenosis. Automatic detection of stenosis allows non-specialists (e.g. nurses, medical assistants, etc.) to measure peak blood velocity and flag patients for further diagnosis. In an embodiment, an ultrasound transducer device of the present invention, a processor, and its associated software can be used as a part of a routine medical visit, such as a physical.

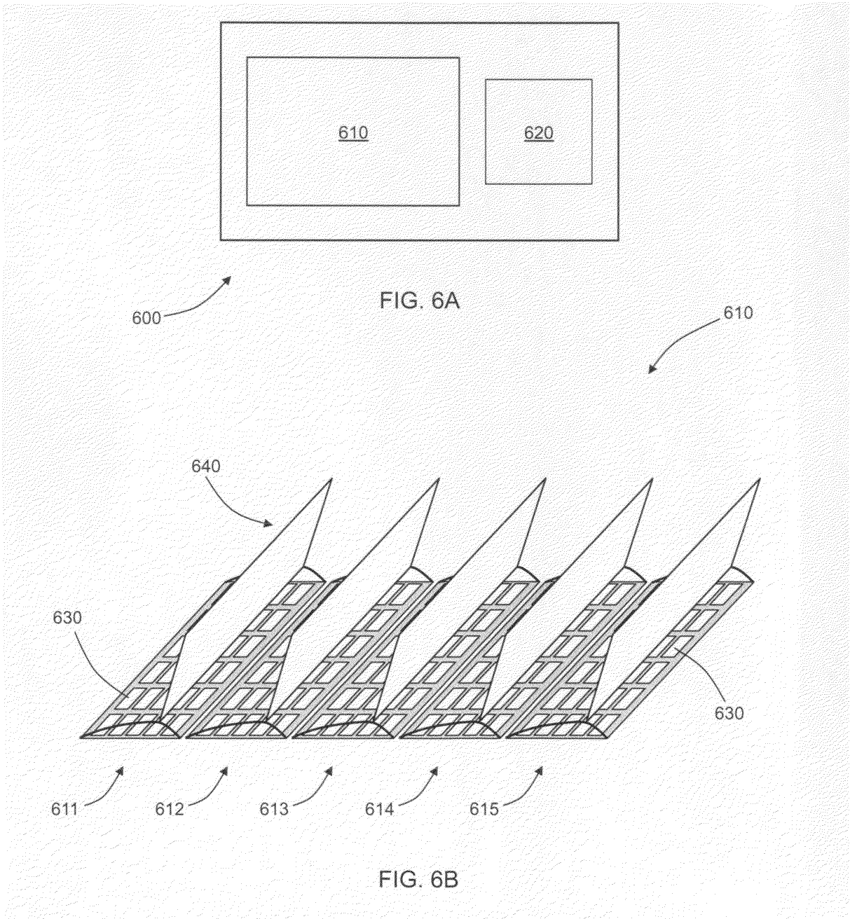

The present invention is also directed to a device for determining the peak blood velocity in a blood vessel. FIG. 6A shows an example device 600 having an array of ultrasound transducers 610 and the electronics 620 necessary to operate the transducers. FIG. 6B shows an exemplary embodiment of the transducer array 610. Preferably, the transducer array 610 is a two-dimensional rectangular array having a plurality of transducer elements 630. In other embodiments, the array can have another configuration, including a circular array, a one-dimensional array, an annular ring array, etc. In a preferred embodiment, the transducer elements 630 are capacitive micromachined ultrasound transducers (CMUTs). In an exemplary embodiment, the ultrasound transducer comprises multiple one-dimensional arrays implemented on a single substrate, wherein each of the one-dimensional arrays images one of the planar cross-sections.

In a preferred embodiment, the transducer array 610 is divided into a number of transducer sub-arrays 611-615. Each of the sub-arrays measures one of the ultrasound images. In other words, the transducer array is divided so that each sub-array is dedicated to measure a single planar cross-section of the blood vessel. By dividing the transducer array 610, the electronics 620 can be simplified. In an embodiment, the transducer array 610 includes multiple acoustic lenses 640, wherein each of the acoustic lenses 640 corresponds with one of the sub-arrays 611-615. An acoustic lens 640 determines the transect angle of the planar cross-section imaged by its corresponding sub-array. As described above, a transect angle less than about 45 degrees is preferred. In an embodiment, the transducer array 610 includes asymmetric acoustic lenses to set the transect angle.

The ultrasound transducer of the present invention can have any operating frequency. Generally, imaging resolution improves with greater operating frequency. However, higher frequencies are more limited by penetration depths than lower frequencies. In a preferred embodiment that can be used for carotid imaging, the transducers of device 600 operate at a frequency range within approximately 5-15 MHz. In an embodiment, the transducer elements 630 of the array 610 are spaced based on the acoustic wavelength corresponding to the operating frequency of the transducers. In particular, the element spacing can be about half of the acoustic wavelength to prevent unwanted grating lobe artifacts in the ultrasound images. In another embodiment, the operating frequency of the transducers is approximately 7 MHz, where the acoustic wavelength in tissue is approximately 200 μm. It is noted that the transducers can operate at operating frequencies outside of the indicated range and can have any element spacing.

Further details of the transducer array, CMUTs, and electronics of the device can be found in U.S. Provisional Patent Application 61/068,004 filed Mar. 3, 2008, which is incorporated herein by reference.

As one of ordinary skill in the art will appreciate, various changes, substitutions, and alterations could be made or otherwise implemented without departing from the principles of the present invention, e.g. the present invention can be applied to finding peak velocity of any fluid in any vessel and is not limited to blood velocity in blood vessels. Accordingly, the scope of the invention should be determined by the following claims and their legal equivalents.

What is claimed is:

1. A method for determining peak velocity of fluid flowing in a blood vessel, said method comprising:
(a) measuring a set of spatially proximate ultrasound images along said blood vessel, wherein said set of ultrasound images are measured with an ultrasound transducer, wherein each of said ultrasound images is of a planar cross-section of said blood vessel, and wherein each of said ultrasound images provides a Doppler-generated velocity map of said fluid in said planar cross-section;
(b) determining an uncorrected peak velocity of said fluid, wherein said uncorrected peak velocity is located at or near one of said planar cross-sections;
(c) identifying a high velocity center in each of a plurality of said ultrasound images;
(d) calculating a Doppler angle based on said high velocity centers of said ultrasound images; and
(e) correcting said peak velocity based on said calculated Doppler angle.

2. The method as set forth in claim 1, wherein said ultrasound transducer comprises a two-dimensional transducer array, wherein said two-dimensional transducer array is divided into a number of transducer sub-arrays, wherein each of said sub-arrays measures one of said ultrasound images of said set of ultrasound images.

3. The method as set forth in claim 2, wherein said ultrasound transducer comprises multiple acoustic lenses, wherein each of said acoustic lenses corresponds with one of said sub-arrays, and wherein each of said acoustic lenses allows said planar cross-section imaged by said corresponding sub-array to transect said blood vessel at a transect angle.

4. The method as set forth in claim 3, wherein said transect angle is less than approximately 45 degrees.

5. The method as set forth in claim 1, wherein said planar cross-sections of said set of ultrasound images are approximately parallel.

6. The method as set forth in claim 1, wherein said ultrasound transducer comprises an array of capacitive micromachined ultrasound transducers.

7. The method as set forth in claim 1, wherein a processor automatically calculates said Doppler angle.

8. The method as set forth in claim 1, further comprising fitting a curve based on said identified high velocity centers of said ultrasound images, wherein said fitted curve represents the direction of said fluid flow in said blood vessel.

9. The method as set forth in claim 8, wherein said blood vessel bifurcates, wherein multiple high velocity centers are identified in each of at least one of said ultrasound images, and wherein one or more curves are fitted to represent said bifurcation.

10. The method as set forth in claim 1, wherein said high velocity center of each of said ultrasound images is identified based on a velocity-thresholded centroid, a velocity center of mass, or a location having the approximately highest velocity in said velocity map of the same of said ultrasound images.

11. The method as set forth in claim 1, further comprising moving said ultrasound transducer approximately along said blood vessel, wherein said movement is to approximately locate said uncorrected peak velocity.

12. The method as set forth in claim 1, further comprising correlating said corrected peak velocity with an amount of stenosis in said blood vessel.

13. The method as set forth in claim 1, wherein said blood vessel is a carotid artery.

14. A device for determining peak velocity of fluid flowing in a blood vessel, said device comprising an array of ultrasound transducers,
wherein said array is divided into multiple sub-arrays, wherein each of said sub-arrays produces a planar cross-sectional ultrasound image of said blood vessel, wherein each of said ultrasound images provides a velocity map of said fluid in said planar cross-section,
wherein an uncorrected peak velocity of said fluid is determined from said ultrasound images, wherein said uncorrected peak velocity is located at or near one of said planar cross-sections produced by said sub-arrays,
wherein a high velocity center is identified in each of said ultrasound images,
wherein a Doppler angle is calculated based on said high velocity centers of said ultrasound images, and
wherein said peak velocity is corrected based on said calculated Doppler angle.

15. The device as set forth in claim 14, further comprising multiple acoustic lenses, wherein each of said acoustic lenses corresponds with one of said sub-arrays, and wherein each of said acoustic lenses allows said planar cross-section imaged by said corresponding sub-array to transect said blood vessel at a transect angle.

16. The device as set forth in claim 14, wherein said planar cross-sections of said blood vessel imaged by said sub-arrays are approximately parallel.

17. The device as set forth in claim 14, wherein said ultrasound transducer array comprises an array of capacitive micromachined ultrasound transducers.

18. The device as set forth in claim 14, wherein said ultrasound transducers operate at a frequency ranging from approximately 5 MHz to approximately 15 MHZ.

19. The device as set forth in claim 14, wherein said ultrasound transducers have an operating frequency, wherein an acoustic wavelength in tissue corresponds with said operating frequency of said ultrasound transducers, and wherein the elements of said array of ultrasound transducers are spaced at approximately half of said acoustic wavelength.

20. The device of claim 14, further comprising a processor, wherein said processor automatically calculates said Doppler angle based on said high velocity centers of said ultrasound images.
